SEQUENCE LISTING (25 sequences)

SEQ ID NO: 1 - protein, 32 aa, Artificial Sequence
  N-terminal hydrophilic domain of the oleosin from hazelnut
MADRPQQLQVHPQRGHGHYEGGIKNQRGGGPS

SEQ ID NO: 2 - protein, 49 aa, Artificial Sequence
  C-terminal hydrophilic domain of the oleosin from hazelnut
NYLRCASQSLPREMDQAKRRMQDMAAFVGQKTREVGQEIQSRAQEGRRT

SEQ ID NO: 3 - protein, 19 aa, Artificial Sequence
  N-terminal hydrophilic domain of the oleosin from walnut
MAEHQQQSQHPAQQPRSHQ

SEQ ID NO: 4 - protein, 47 aa, Artificial Sequence
  C-terminal hydrophilic domain of the oleosin from walnut
YRYMTGRQPPGTEQIDQARMKLASKAREMKDKAEQFGQQHVSGAQSS

SEQ ID NO: 5 - protein, 105 aa, Artificial Sequence
  Consensus sequence I of oleosins
XPXXXXXXXXXXXXXXXXXLLXXXGXXLXXXXXXLXXXXPXXXXFSPXXVPAXXXXXXXXXXFLXSGXXGXXXXXXXXWXXXXXXXXXXXXXXXXXXAXXXXXXX

SEQ ID NO: 6 - protein, 105 aa, Artificial Sequence; identical to SEQ ID NO: 5
  Consensus sequence II of oleosins

SEQ ID NO: 7 - protein, 10 aa, Artificial Sequence
  C-terminal sequence motif I of oleosins
LXXKAREXKD

SEQ ID NO: 8 - protein, 6 aa, Artificial Sequence
  C-terminal sequence motif II of oleosins
VGXKTX

SEQ ID NO: 9 - protein, 12 aa, Artificial Sequence
  Consensus sequence I for hydrophobic domain of oleosins
PXXXXFSPXXVP

SEQ ID NO: 10 - protein, 12 aa, Artificial Sequence
  Consensus sequence II for hydrophobic domain of oleosins
PXXXXFSPXXVP

SEQ ID NO: 11 - protein, 235 aa, Artificial Sequence
  Consensus sequence I of caleosins
XXXXXXXXXXXXXXXXXXXAXXAXXTXXRXXRXDLXXXXPKPYXXRXXXAPDXXXXXGTXXHXXXXXSVLQQHXXFFDXDXXGIIYPXETXXGXRXXGFNXXXSXIXXXXXXXXXSYXTLPXWXPSXXFXIXXXNIHXXKHGSDSXXYDXEGRXXPXNXEXXFSKXAXTXPDXLXXXELWXXTXXNRXAXDXFGWXAXKXEWXXLYXLAXDXXGXLXKEAXRRCXDGSLFEYCAK

SEQ ID NO: 12 - protein, 235 aa, Artificial Sequence; identical to SEQ ID NO: 11
  Consensus sequence II of caleosins

SEQ ID NO: 13 - protein, 11 aa, Artificial Sequence
  Consensus sequence I for hydrophobic domain of caleosins
PXWXPSXXFXI

SEQ ID NO: 14 - protein, 10 aa, Artificial Sequence
  Consensus sequence II for hydrophobic domain of caleosins
PXWXPSXXFX

SEQ ID NO: 15 - protein, 242 aa, Artificial Sequence
  Consensus sequence I of steroleosins
XLXXXXXNXXXPXXXXXXXXXXXPXXXXXXXXXXXXXXXXXXXXXXXKVVXITGASXXIGEXXAYXYAXXXAXLXLXAXRXXXLXXXXXXARXXGXXXVXXXXADVXKXXXCXXXXXXXXXXXGRXDHLVNXXXXXXXXXXXXXXXXXXXXXXXXXNFWGXXYXTXXAXXXLXXXXGXXXXXXSXXXWXXXPRXXXYXAXKXAXXXXXETLRXEXXXXXXXTXXTXGXXXXEXXXGKXXXXX

SEQ ID NO: 16 - protein, 242 aa, Artificial Sequence; identical to SEQ ID NO: 15
  Consensus sequence II of steroleosins

SEQ ID NO: 17 - protein, 13 aa, Artificial Sequence
  Consensus sequence I for hydrophobic domain of steroleosins
PXXXXXXXXXXXP

SEQ ID NO: 18 - protein, 12 aa, Artificial Sequence
  C-terminal sequence motif I of steroleosins
XXXRXXXXXXXP

SEQ ID NO: 19 - protein, 46 aa, Artificial Sequence
  C-terminal sequence motif II of steroleosins
XXXXAXXXXXXXXRXXXXXXXPXWXXXXXXXXXXXPXXXXWXXXXX

SEQ ID NO: 20 - protein, 15 aa, Artificial Sequence
  C-terminal sequence motif III of steroleosins
KKXLDXXGXXXXXYP

SEQ ID NO: 21 - protein, 13 aa, Artificial Sequence
  C-terminal sequence motif IV of steroleosins
XXLXXXXGXXXXS

SEQ ID NO: 22 - protein, 6 aa, Artificial Sequence
  Exemplary linker sequence
TLAGSV

SEQ ID NO: 23 - protein, 87 aa, Artificial Sequence
  Exemplary construct derived from Cor a
MADRPQQLQVHPQRGHGHYEGGIKNQRGGGPSTLAGSVNYLRCASQSLPREMDQAKRRMQDMAAFVGQKTREVGQEIQSRAQEGRRT

SEQ ID NO: 24 - protein, 176 aa, Artificial Sequence
  Sequence of Ara h 15 with N-terminal His-tag
HHHHHHIEGRMSDQTRTGYGGGGSYGSSYGGGGTYGSSYGTSYDPSTNQPIRQAIKFMTASTIGVSFLILSGLILTGTVIGLIIATPLLVIFSPILVPAAITLALAAGGFLFSGGCGVAAIAALSWLYSYVTGKHPAGSDRLDYAKGVIADKARDVKDRAKDYAGAGRAQEGTPGY

SEQ ID NO: 25 - protein, 129 aa, Artificial Sequence
  Sequence of Ara h 15 with N-terminal His-tag without hydrophobic domain
HHHHHHIEGRMSDQTRTGYGGGGSYGSSYGGGGTYGSSYGTSYDPSTNQPIRQAIKFMTASTTPLLVIFSPILVPALSWLYSYVTGKHPAGSDRLDYAKGVIADKARDVKDRAKDYAGAGRAQEGTPGY

(19) United States
(12) Patent Application Publication
(10) Pub. No.: US 2016/0320407 A1
(43) Pub. Date: Nov. 3, 2016

(54) DETECTION OF NUT ALLERGIES

(71) Applicant: EUROIMMUN Medizinische Labordiagnostika AG, Lübeck (DE)

(72) Inventors: Anna Ehlers, Lübeck (DE); Yvonne Michel, Hamburg (DE); Stefanie Rohwer, Kalkhorst (DE); Waltraud Suer, Buchholz (DE); Mandy Unger, Hamburg (DE)

(21) Appl. No.: 15/143,011

(22) Filed: Apr. 29, 2016

(30) Foreign Application Priority Data
Apr. 30, 2015 (EP) 15001277.1

(51) Int. Cl. G01N 33/68; G01N 33/543; C07K 14/415

(52) U.S. Cl. CPC G01N 33/6854; C07K 14/415; G01N 33/543; C07K 2319/00; G01N 2800/24; A61K 39/00

(57) ABSTRACT

The present invention relates to a polypeptide comprising an antigenic fragment having a sequence of at least seven amino acids from a hydrophilic domain of an oil body-associated protein, wherein the polypeptide comprises a truncated hydrophobic domain of the oil body-associated protein or does not comprise such a domain, and wherein the oil body-associated protein is selected from the group comprising oleosin, caleosin and steroleosin, to a pharmaceutical composition or a vaccine comprising the polypeptide, to a complex comprising the polypeptide and an antibody bound thereto, to a medical or diagnostic device comprising the polypeptide, to a nucleic acid encoding the polypeptide or a cell comprising said nucleic acid, to a test kit comprising the polypeptide, and to a method for detecting an antibody which binds to the polypeptide.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 310159_409_SEQUENCE_LISTING.txt. The text file is 43.3 KB, was created on Apr. 28, 2016, and is being submitted electronically via EFS-Web.

### BACKGROUND

1. Technical Field

The present invention relates to a polypeptide comprising an antigenic fragment having a sequence of at least seven amino acids from a hydrophilic domain of an oil body-associated protein, wherein the polypeptide comprises a truncated hydrophobic domain of the oil body-associated protein or does not comprise such a domain, and wherein the oil body-associated protein is selected from the group comprising oleosin, caleosin and steroleosin, to a pharmaceutical composition or a vaccine comprising the polypeptide, to a complex comprising the polypeptide and an antibody bound thereto, to a medical or diagnostic device comprising the polypeptide, to a nucleic acid encoding the polypeptide or a cell comprising said nucleic acid, to a test kit comprising the polypeptide, and to a method for detecting an antibody which binds to the polypeptide.

2. Description of the Related Art

The “Big 8” of allergy sources includes not only milk, eggs, soy, wheat, fish, shellfishes and peanuts, but also seeds and kernels, and is responsible for up to 90% of allergic reactions to foodstuffs. With an annual import volume of 200 megatons, hazelnut and walnut are the most relevant nut types in Germany (Hanson, R. and A. Medina, 2012, Tree nuts annual 2012, USDA Foreign Agriculture Service).

An allergic reaction is a hypersensitivity of the immune system to a nonpathogenic substance from the environment, resulting in the development of clinical disease symptoms (von Pirquet, C. 1906, “Allergie” [Allergy], Münchener medizinische Wochenschrift [Munich weekly medical journal] 53, pages 1457-8). Especially in highly developed industrialized countries, there are, with a quarter of the population, more and more people sensitized to one or more allergens (Galli, S. J., M. Tsai and A. M. Piliponsky 2008, “The development of allergic inflammation”, Nature 454.7203, pages 445-54). The symptoms that occur range from urticaria through to emesis and diarrhea and up to anaphylactic shock.

For the unambiguous diagnosis of a nut allergy, the development of a component-specific in vitro allergy test is of great interest. By using individual allergens, it is possible to identify primary sensitizations and cross-reactivities, the result being the creation of a sensitization pattern. By means of such a pattern, it is possible to carry out an improved risk assessment which leads to an optimized therapeutic approach (Treudler, R. 2012, “In-vitro-Allergiediagnostik aktuell” [Current in vitro allergy diagnostics], Journal of the German Society of Dermatology 2.10, pages 89-100).

The hazelnut, Corylus avellana, belongs to the birch family (Betulaceae). Zuidmeer-Jongejan et al. (“Oil body-associated hazelnut allergens including oleosins are underrepresented in diagnostic extracts but associated with severe symptoms”, Clinical and Translational Allergy 4.1, pages 1-10) demonstrated that, in allergen extracts, the oleosins identified as allergens (Cor a 12 and Cor a 13) are underrepresented because of the preparation process. Oleosins are suspected of triggering severe systemic reactions, right up to anaphylactic shock (Kleine-Tebbe, J. et al., 2009, in-vitro-Diagnostik und molekulare Grundlagen von IgE-vermittelten Nahrungsmittelallergien [IgE-mediated food allergies—in vitro diagnostics and molecular bases], Jan. 18, 2015, Gesellschaft für Pädiatrische Allergologie und Umweltmedizin e. V. [German registered society for pediatric allergology and environmental medicine]). Nut oleosins have so far been little studied, since the isolation thereof is a major challenge because of the long hydrophobic segment (Hsieh, K. and A. H. Huang 2004, “Endoplasmic Reticulum, Oleosins, and Oils in Seeds and Tapetum Cells”, Plant Physiology 136.3, pages 3427-34). In commercial tests, there is to date still no use of oil body-associated nut allergens.

The English walnut (Latin: Juglans regia), like the pecan, belongs to the walnut family (Latin: Juglandaceae) and is the walnut type that is consumed with preference in North America (Roux, K. H., S. S. Teuber and S. K. Sathe (2003). “Tree nut allergens”. International Archives of Allergy and Immunology 131.4, pages 234-44). In 2012, 239.5 megatons were consumed within the European Union (EU) (Hanson, R. and A. Medina (2012). Tree nuts annual 2012. USDA Foreign Agriculture Service). Major walnut allergens that have been identified are a 2S albumin (Jug r 1), a vicilin (Jug r 2), an LTP (Jug r 3) and an 11S globulin (Jug r 4). A sensitization to major walnut allergens can lead to severe systemic reactions, meaning that consumption can trigger an anaphylactic shock (Agreiter, C. (2013). “Purification of seed storage proteins from tree nuts and peanut and their range of cross-reactivity—implication for the nut allergic patient”. Thesis for master's degree. University of Vienna). Owing to a 75% homology between Jug r 2 and Ara h 1 (peanut vicilin), a sensitization to Jug r 2 can lead to a cross-reactivity to peanuts. Cross-reactivities between walnut and peach likewise occur, since the LTP Jug r 3 has a high homology to the LTP from peach (Roux, K. H., S. S. Teuber and S. K. Sathe (2003). “Tree nut allergens”. International Archives of Allergy and Immunology 131.4, pages 234-44).

The pecan, Carya illinoinensis, belongs to the walnut family. Pecan consumption in the USA in 2013 was estimated to be 440 million pounds (Nature's Finest Food, Ltd., 2013, US Domestic Pecan Consumption Rises. Feb. 7, 2015). In 2010, Sharma identified the allergens Car i 1, Car i 2 and Car i 4 and prepared said allergens recombinantly. In the case of the allergens Car i 1, a 2S albumin, and Car i 4, an 11S globulin, digestion-resistant epitopes were identified. In the case of the 2S albumin Car i 1, five highly reactive, linear epitopes were identified, and these are possibly the cause of Car i 1 being able to trigger severe allergic reactions (Sharma G. M. et al., 2011, “Cloning and characterization of 2S albumin, Car i 1, a major allergen in pecan”. Journal of Agricultural and Food Chemistry 59.8, pages 4130-9). The allergen Car i 4 likewise has highly reactive, linear epitopes, which are located on the acidic subunit of the 11S globulin (Sharma G. M. et al., 2011b, “Cloning and Characterization of an 11S Legumin, Car i 4, a Major Allergen in Pecan”, Journal of Agricultural and Food Chemistry 59.17, pages 9542-52). Inhibition assays demonstrated that specific IgE antibodies against walnut cross-react with pecan (Teuber, S. S., S. K. Sathe, K. H. Roux and W. R. Petersen, 2000, “Cross-reactivity of walnut and other tree nuts by IgE immunoblot inhibition”, Journal of Allergy and Clinical Immunology 105.1, page 140).

An allergy can be diagnosed using various principles of testing. Besides medical history, in which a physician collects information about the patient by means of specific questions and determines indications of possible causes of allergy, multiple examination methods are usually consulted in combination to provide clarification. These include using in vivo skin tests and in vitro laboratory tests. For example, in the case of the in vivo skin prick test, allergenic extracts or else individual allergens are applied to the skin of the patient and the epidermis is lightly scratched through the drop. A positive reaction can be identified by the formation of wheals (Sicherer et al., 2009, “Allergy Frontiers: Diagnosis and Health Economics.”, Springer Verlag).

In vitro, it is possible to collect information at the molecular level by the detection of allergen-specific antibodies in the blood of the patient (Renz et al., 2010, “In-vitro-Allergiediagnostik—Leitlinie der Deutschen Gesellschaft fúr Allergologie und klinische Immunologie (DGAKI) unter Beteiligung des Ärzteverbandes Deutscher Allergologen (ÄDA), der Gesellschaft für Pädiatrische Allergologie und Umweltmedizin (GPA) und der Deutschen Dermatologischen Gesellschaft (DDG)” [In vitro allergy diagnostics—guideline from the DGAKI (German society for allergology and clinical immunology) in cooperation with the ÄDA (German allergologist medical association), the GPA (society for pediatric allergology and environmental medicine) and the DDG (German dermatological society)], Allergo Journal, 19, pages 110-28). In this connection, a sensitization of the patient is detected by immobilizing allergen extracts or individual allergens on a solid phase and then incubating them with the patient serum. For the purposes of detection, specific antibodies bound to the allergen can be detected using a labeled secondary antibody (Lutmann et al., 2009, “Der Experimentator—Immunologie” [The experimenter—immunology], Spektrum Akademischer Verlag). In recent years, the use of individual allergens, recombinant as well as native and isolated, in serological tests has become established. The method referred to as component-specific diagnostics allows a more differentiated statement to be made in relation to the sensitization pattern for a polyvalent allergy (Hemmer, W. (2009). “Allergiediagnostik—Extrakte oder Moleküle?” [Allergy diagnostics—extracts or molecules?] Wener Medizinische Wochenschrift [Vienna weekly medical journal] 6.8, pages 9-11).

Further in vitro methods include cellular assays, in which allergic reactions are examined in a functional, cellular system. Examples include the determination of the in vitro release of histamine from peripheral leukocytes, the degranulation of basophilic leukocytes, the basophil activation test and the lymphocyte transformation test (Ring, J. In: “Angewandte Allergologie” [Applied allergology], Urban & Vogel, Munich, 2004, 3rd edition).

The gold standard of allergy diagnostics is considered to be the provocation test, which makes it possible to make an unambiguous diagnosis. This involves supplying the patient with an increasing dose of allergen until symptoms are displayed (Bock et al., “Double-blind, placebo-controlled food challenge (DBPCFC) as an office procedure: a manual.”, Journal of Allergy and Clinical Immunology, 1988; 82.6, pages 986-97, Niggemann et al., “Standardisierung von oralen Provokationstests bei Verdacht auf Nahrungsmittelallergie” [Standardization of oral provocation tests for suspected food allergy], Allergo Journal 2011; 20:149-60, Riechelmann et al., 2002, “Durchführung des nasalen Provokationstests bei Erkrankungen der oberen Atemwege” [Performance of the nasal provocation test for diseases of the upper airways], Allergo Journal 2002; 11:29-36).

While numerous standard antigens are routinely used for detecting allergy-indicative IgE, it is a source of regret to the specialist community that oil body-associated proteins have so far not been adequately taken into consideration in the design of routine tests.

The reasons for this include, firstly, the poor isolatability and low stability of such proteins, especially under demanding climatic conditions, which are conventionally isolated as full-length proteins from oil body fractions of nuts (Zuidmeer-Jongehan et al., 2014). In addition, only low amounts can be obtained in this way in batches of poorly reproducible quality. Methods which permit an isolation of sufficiently stable antigen on a large scale are not described in the prior art.

With regard to the quality of tests designed using such reagents, the specialist community expresses its dissatisfaction. There are explicit complaints concerning the low sensitivity and unreliability (Zuidmeer-Jongehan et al., 2014).

Another problem is that the commercially available tests do not allow a reliable statement to be made as to the severity of the allergy of a patient, for example whether a patient is only suffering from an allergy with mild symptoms or whether serious complications up to life-threatening complications may occur.

Against this background, there is a considerable need for novel reagents and methods based on oil body-associated proteins for detecting food allergies, especially to plant parts such as seeds and kernels, more precisely nuts.

### SUMMARY

It is an object of the present invention to overcome the stated disadvantages and further disadvantages of the reagents and methods described in the prior art for diagnosing allergies to nuts.

It is a further object to provide reagents and methods for diagnosing allergies to nuts that are advantageous with respect to specificity and/or sensitivity. More particularly, it is intended that the proportion of false positive and/or false negative results be reduced.

It is a further object to provide reagents industrially manufacturable on a routine basis and/or large scale and methods suitable for manufacturing said reagents, and also tests for detecting nut allergies having reproducible properties, which tests can be manufactured on a routine basis and/or large scale.

It is a further object to provide reagents which are as nonsensitive as possible and which function reliably under very extreme climatic conditions.

It is a further object to provide a test, reagents and methods which allow a differentiated diagnosis or prognosis of allergies, more particularly an assessment of the severity of the allergy which a patient is suffering from.

These objects and further objects are achieved by the subject matter of the present application and especially also by the subject matter of the accompanying independent claims, with embodiments being revealed by the dependent claims.

In a first aspect, the object underlying the invention is achieved by a polypeptide comprising an antigenic fragment having a sequence of at least seven amino acids from a hydrophilic domain of an oil body-associated protein, wherein the polypeptide comprises a truncated hydrophobic domain of the oil body-associated protein or does not comprise such a domain, and wherein the oil body-associated protein is selected from the group comprising oleosin, caleosin and steroleosin.

In a first preferred embodiment of the first aspect, the polypeptide comprises
a) an antigenic fragment of at least seven amino acids from an N-terminal hydrophilic domain of an oil body-associated protein and
b) an antigenic fragment of at least seven amino acids from a C-terminal hydrophilic domain of an oil body-associated protein,

wherein a) and b) are fused to one another or are joined to one another by means of a linker, and wherein the oil body-associated protein is selected from the group comprising oleosin and caleosin.

In a second preferred embodiment of the first aspect, which is also one embodiment of the first embodiment, the oil body-associated protein is an oleosin.

In a third preferred embodiment, which is also one embodiment of the first to second embodiments, a) is a sequence from SEQ ID NO:1 and variants thereof and b) is a sequence from SEQ ID NO:2 and variants thereof.

In a fourth preferred embodiment, which is also one embodiment of the first to third embodiments, a) is a sequence from SEQ ID NO:3 and variants thereof and b) is a sequence from SEQ ID NO:4 and variants thereof.

In the fifth preferred embodiment, which is also one embodiment of the first to fourth embodiments, the polypeptide is immobilized, preferably on a solid support, even more preferably on a microbead.

In a second aspect, the object is achieved by a pharmaceutical composition or a vaccine comprising the polypeptide.

In a third aspect, the object is achieved by a complex comprising the polypeptide and an antibody, preferably of the IgE class, bound thereto.

In a fourth aspect, the object is achieved by a medical or diagnostic device comprising the polypeptide according to the invention.

In a preferred embodiment of the fourth aspect, said device is a test strip, a biochip or an electrophoresis gel.

In a fifth aspect, the object is achieved by a nucleic acid encoding the polypeptide according to the invention or the cell comprising said nucleic acid.

In a sixth aspect, the object is achieved by a test kit comprising the polypeptide according to the invention, optionally additionally comprising a means for detecting the complex according to the invention.

In a seventh aspect, the object is achieved by a method comprising the step of detecting an antibody which binds to the polypeptide according to the invention in a sample from a mammal, preferably a human, containing antibodies, preferably of the IgE class.

In a first preferred embodiment of the seventh aspect, the mammal exhibits at least one symptom of an allergy and/or it is suspected that the mammal suffers from an allergy, preferably to nuts.

In a second preferred embodiment of the seventh aspect, which is also one embodiment of the first aspect, the method comprises the steps of
a) providing a sample from a mammal,
b) contacting the sample with the polypeptide according to the invention or the medical device under conditions compatible with the formation of a complex between the polypeptide and an antibody, and
c) detecting a complex formed in step b).

### BRIEF DESCRIPTION OF THE DRAWINGS

FIG. 1 shows an SDS-PAGE analysis of the IMAC-purified recombinant Cor a 12. The analysis was carried out by means of a 4-12% bis-Tris gel and subsequent Coomassie staining. Lane 1: broad range marker, lane 2: purified, recombinant Cor a 12 (nonreduced), lane 3: purified, recombinant Cor a 12 (reduced).

FIG. 2 shows a Cor a 12-coated line blot.

FIG. 3 shows a reactivity analysis of the hazelnut Cor a 12 polypeptide according to the invention in the Euroline system. The reactivity analysis was done using sera from hazelnut allergy sufferers. The evaluation of bound antibodies from the patient sera was done using the EAST reference system.

FIG. 4 shows a nitrocellulose membrane coated with constructs in accordance with SEQ ID NO:25 and SEQ ID NO:24, having three different patient sera applied to it. Serum 1: patient 1 with clinical symptoms upon consumption of nuts, Serum 2: patient 2, pollen allergy sufferer without specific IgE against nuts, Serum 3: patient 3, pollen allergy sufferer without specific IgE against nuts.

### DETAILED DESCRIPTION

The present invention is based on the surprising finding by the inventors that the disadvantages of the reagents and methods described in the prior art can be overcome by using the polypeptide according to the invention.

Without wishing to be tied to any theory, the inventors suspect that further polypeptides bind to the hydrophobic domain of oil body-associated proteins, which polypeptides contaminate oil body-associated proteins used as antigens for diagnostic tests. Both allergy-specific antibodies in the blood of patients and further antibodies bind to these contaminants, impairing specificity, for example in the form of false positive results.

Said contaminants cannot bind to the recombinant oil body-associated proteins according to the invention having a truncated hydrophobic domain, since the binding sites are absent because of the truncation. The risk of false positive results is therefore reduced if diagnostic tests are designed on the basis of such recombinant polypeptides.

Also without wishing to be tied to any theory, the inventors suspect that, during isolation, native oil body-associated proteins form a conformation or undergo physicochemical reactions which lead(s) to the concealment of allergy-relevant epitopes, and this can impair sensitivity, for example because allergy-specific antibodies in samples cannot bind to the antigen.

Also without wishing to be tied to any theory, the inventors further suspect that the truncation brings about a physicochemical stabilization of the recombinant antigen in comparison with the native full-length protein.

The present invention provides recombinant polypeptides derived from oil body-associated proteins, selected from the group comprising oleosins, caleosins and steroleosins, having a truncated hydrophobic domain.

What oleosins, caleosins and steroleosins have in common is that they are part of the oil body-associated proteins in plants. The native full-length proteins of said proteins are divided into a hydrophobic domain and a hydrophilic domain connected C-terminally thereto. Whereas the steroleosins do not have any further domains, oleosins and caleosins additionally have an N-terminal hydrophilic domain.

The term “oleosin”, as used here, is understood to mean a polypeptide having the sequence SEQ ID N0:5, preferably SEQ ID N0:6, even more preferably in combination with a sequence from the group comprising SEQ ID N0:7 and SEQ ID N0:8, and also variants of the sequences in each case. In a preferred embodiment, oleosin is an oleosin from the group comprising those with sequences represented by the database codes Q647G5 (Ara h 10), Q45W87 (Ara h 11), 75148112 (Q84T21, Cor a 12), 75298094 (Q84T91, Cor a 13), G8H6H8 (Jug r Oleosin-1), G8H6H9 (Jug r Oleosin-2), Q9FUJ9 (Ses i 4), Q9XHP2 (Ses i 5) and 004925 (Ses i Oleosin), particularly preferably Q647G5, and also variants of the sequences in each case. All the database codes correspond to those sequences that could be retrieved from the particular databases on Apr. 30, 2015.

In the case of an oleosin, the hydrophobic domain is characterized in that it comprises SEQ ID N0:9 and variants thereof and also the 32, more preferably 29, more preferably 27, more preferably 24, more preferably 20, more preferably 18, more preferably 12, more preferably 0 amino acids lying N-terminally therebefore, and also the 28, more preferably 25, more preferably 15, more preferably 10, more preferably 0 amino acids lying C-terminally thereafter. More preferably, in the case of an oleosin, the hydrophobic domain is characterized in that it comprises SEQ ID N0:10 and variants thereof and also the 32, more preferably 29, more preferably 27, more preferably 24, more preferably 20, more preferably 18, more preferably 12, more preferably 0 amino acids lying N-terminally therebefore, and also the 28, more preferably 25, more preferably 15, more preferably 10, more preferably 0 amino acids lying C-terminally thereafter.

The term “caleosin”, as used here, is understood to mean a polypeptide having the sequence SEQ ID N0:11, preferably SEQ ID N0:12, and also variants of the sequences in each case. In a preferred embodiment, caleosin is a caleosin from the group comprising those with sequences represented by the database codes Q9SQ57-1 (peroxy Ses), 081270-1 (peroxy ara t (Arapidopsis thaliana)), Q9FLN9-1 (PXG2 Ara t), 023959-1 (Gly m Call), M5WJM8-1 (Pru p Call), particularly preferably Q9SQ57-1, and also variants of the sequences in each case.

In the case of a caleosin, the hydrophobic domain is characterized in that it comprises SEQ ID N0:13 and variants thereof and also the 23, more preferably 20, more preferably 10, more preferably 0 amino acids lying N-terminally therebefore, and also the 7, more preferably 5, more preferably 3, more preferably 2, more preferably 0 amino acids lying C-terminally thereafter. More preferably, in the case of a caleosin, the hydrophobic domain is characterized in that it comprises SEQ ID NO:14 and variants thereof and also the 23, more preferably 20, more preferably 10, more preferably 0 amino acids lying N-terminally therebefore, and also the 7, more preferably 5, more preferably 3, more preferably 2, more preferably 0 amino acids lying C-terminally thereafter.

The term “steroleosin”, as used here, is understood to mean a polypeptide having the sequence SEQ ID NO:15, preferably SEQ ID NO:16, even more preferably in combination with a sequence from the group comprising SEQ ID NOS:18, 19, 20 and 21, and also variants of the sequences in each case. In a preferred embodiment, steroleosin is a steroleosin from the group comprising those with sequences represented by the database codes A7LB59 (Ara h Ster), Q93W57 (Ses i Ster 1), C3S7G7 (Bra n Ster 1), C3S7G9 (Bra n Ster 2), Q8LKV5 (Ses i Ster 2), A7LB60 (Ara h Ster 2), C3S7H0 (Bra n Ster 3) and C3S7H1 (Bra n Ster 4), particularly preferably A7LB59, and also variants of the sequences in each case.

In the case of a steroleosin, the hydrophobic domain is characterized in that it comprises SEQ ID NO:17 and variants thereof and also all amino acids lying N-terminally therebefore, and also the 7, more preferably 5, more preferably 3, more preferably 2, more preferably 0 amino acids lying C-terminally thereafter.

In a preferred embodiment, the term “hydrophilic domain” is understood to mean any domain within an oleosin, caleosin or steroleosin beyond the hydrophobic domain. In the case of an N-terminal hydrophilic domain, this is the domain lying before the hydrophobic domain in the primary sequence. In the case of a C-terminal hydrophilic domain, this is the domain lying after the hydrophobic domain in the primary sequence.

The polypeptide according to the invention comprises an antigenic fragment having a sequence of at least seven amino acids from a hydrophilic domain of an oil body-associated protein, wherein the polypeptide comprises a truncated hydrophobic domain of the oil body-associated protein or does not comprise such a domain, and this means, in a preferred embodiment, the absence of the entire hydrophobic domain from the corresponding wild-type protein. The truncated hydrophobic domain has, in order of increasing preference, a length of 99%, 95%, 90%, 80%, 70%, 60%, 50%, 40%, 30%, 20%, 10% or 5% of its length in the corresponding wild-type polypeptide. In this connection, the hydrophobic domain is preferably truncated such that the formation of a transmembrane domain does not occur. In a preferred embodiment, the term “antigenic fragment” from a polypeptide is to be understood to mean an amino acid sequence taken therefrom having at least 7, 10, 15, 20, 30 or 40 successive amino acid residues, which specifically binds to antibodies of the IgE class from patients having a corresponding allergy.

In a preferred embodiment, the polypeptide according to the invention additionally comprises a polypeptide which increases solubility in aqueous solution. A person skilled in the art is aware of numerous suitable fusion proteins, for example MPB, GST, protein G B1 domain, thioredoxin, NusA, ubiquitin, SUMO and T7 gene 10. This solubility-increasing polypeptide can be arranged N-terminally or C-terminally in relation to the antigenic fragments or among the antigenic fragments; preferably, it lies N-terminally.

In a preferred embodiment, the term “linker”, as used here, is understood to mean a flexible chain of amino acids, preferably of a length of from 1 to 100, from 2 to 80, from 3 to 50 or from 4 to 40 amino acids. The purpose of the linker is to join fragments in a covalent manner without restricting the steric accessibility thereof. One possible linker sequence is represented in SEQ ID NO:22, and an exemplary total construct is thus represented in SEQ ID NO:23.

The teaching of the present invention can be carried out not only by using macromolecules having the exact amino acid sequence or nucleic acid sequence to which reference is made here, for example antigenic fragments of oil body-associated proteins, but also by using a variant of such macromolecules, which can be obtained by deletion, addition or substitution of one or more than one amino acid or nucleic acid. In a preferred embodiment, the term “variant” of a nucleic acid sequence or amino acid sequence, as used here, means a different sequence which has a homology of, in order of increasing preference, 70%, 75%, 80%, 85%, 90%, 92%, 94%, 96%, 98%, 99% or a higher percent with respect to the first-mentioned nucleic acid sequence or amino acid sequence, and preferably amino acids other than the ones important for biological activity are changed in a corresponding expressed amino acid sequence. In this connection, the term “homology” means either amino acid identity or conservative substitution for a different amino acid, preferably identity. In a more preferred embodiment, a conservative substitution means in this case that an amino acid from the following groups is substituted for a different amino acid from the same group: hydrophobic amino acids (V, L, I), hydroxyl group-containing amino acids (T, S), acidic amino acids (D, E), amidic amino acids (N, Q), basic amino acids (K, R) and aromatic amino acids (F, V.

In a more preferred embodiment of the present invention, the variant of an amino acid sequence or nucleic acid sequence has, preferably in addition to the abovementioned sequence homology, substantially the same biological activity of the wild-type molecule or of the original molecule. For example, a variant of a polypeptide which specifically binds to a particular antibody has the same or substantially the same binding specificity as the wild-type molecule. In a particular embodiment, the term “substantially the same binding specificity” means a dissociation constant which differs by not less than a factor of 1000, more preferably 500, even more preferably 100, even more preferably 50, even more preferably 20, even more preferably 10, even more preferably 2 from the dissociation constant for the binding reaction between the same antibody with the first-mentioned, original sequence.

The variant of the nucleic acid sequence or amino acid sequence has the same length of the first-mentioned, original sequence or a fragment thereof, for example a fragment having at least 7, more preferably 10, 20, 30, 40, 50, 60, 70, 80, 90, 100, 120, 150 or 200 amino acids, it being possible for the fragment to start at the C-terminus, at the N-terminus or between said termini, preferably at the N-terminus, of a hydrophilic domain lying N-terminally from the hydrophobic domain or it being possible for the fragment to lie at the N-terminus of a hydrophilic domain lying C-terminally from the hydrophobic domain. Preferably, the fragment comprises, in order of increasing preference, 20%, 30%, 40%, 50%, 60%, 70%, 80%, 90%, 95%, 99% or 100% of the amino acids of the particular N-terminal or C-terminal domain.

It is essential that the variant of the polypeptide exhibits biological activity, i.e., to specifically bind to nut allergy-specific antibodies in samples from patients with a nut allergy, preferably to antibodies of the IgE class. By means of sera from patients who suffer from nut allergies and by means of conventional detection techniques, preferably by means of a Western blot, it is possible to verify whether said biological activity is present.

To carry out the present invention, the polypeptide according to the invention can be provided in any form and in any purity level, from tissues or cells expressing the polypeptide, preferably cells overexpressing the polypeptide, crude or enriched lysates of such cells, up to purified and/or isolated polypeptide, which is substantially pure. The polypeptide according to the invention can be an unfolded, chemically synthesized peptide. The polypeptide according to the invention can be a folded polypeptide. In a preferred embodiment, the polypeptide is a recombinant polypeptide, the term “recombinant”, as used here, referring to a polypeptide which has been prepared using gene technology techniques at some stage of the production process, for example by the fusion of a nucleic acid encoding the polypeptide to a relatively strong promoter for overexpression in cells or tissues or by modification of the sequence of the polypeptide or of a nucleic acid itself encoding said polypeptide. A person skilled in the art in the field is familiar with methods comprising gene technology techniques for modifying nucleic acids and polypeptides (see, for example, Sambrook, J., Fritsch, E. F. and Maniatis, T. (1989), Molecular Cloning, CSH or Brown T. A. (1986), Gene Cloning—an introduction, Chapman & Hall) and is capable of preparing and purifying recombinant polypeptides (see, for example, the manuals “Strategies for Protein Purification”, “Antibody Purification”, “Purifying Challenging Proteins”, “Recombinant Protein Purification”, “Affinity Chromatography”, “Ion Exchange Chromatography”, “Gel Filtration (Size Exclusion Chromatography)”, “Hydrophobic Interaction Chromatography”, “Multimodal Chromatography” (2009/2010), published by GE Healthcare Life Sciences, and in Burgess, R. R., Deutscher, M. P. (2009), Guide to Protein Purification). In a preferred embodiment, a polypeptide is pure when at least 60%, 70%, 80%, 90%, 95% or 99% of the total polypeptide in the particular sample consists of the polypeptide, as can be assessed by visual inspection after SDS-PAGE followed by Coomassie blue staining.

If the polypeptide is provided in the form of a recombinant cell, the cell can be a eukaryotic cell, such as a yeast cell, or a prokaryotic cell, such as Escherichia coli. For example, the cell can be an HEK293 cell transfected with the nucleic acid functionally encoding the polypeptide according to the invention. A person skilled in the art in the field is familiar with methods for preparing, transfecting and culturing such cells, as described, for example, in Phelan, M. C. (2001), Basic Techniques in Mammalian Cell Tissue Culture, John Wiley.

The polypeptide used to carry out the teaching according to the invention, and also any variants thereof, are designed so as to exhibit epitopes which are recognized by antibodies in sera from patients who suffer from a disease, preferably from an allergy, more preferably an allergy to plant parts, more preferably to seeds and kernels, even more preferably to nuts.

Such a polypeptide has a segment of at least 7, 8, 9, 10, 11, 12, 20, 25, 30, 40, 50, 60, 70, 80, 90, 100 or more successive amino acids from an oil body-associated protein. A person skilled in the art in the field is familiar with guidelines which can be used to design peptides of sufficient immunogenicity, as described, for example, in Jackson, D. C., Fitzmaurice, C. J., Brown, L. E., Zeng, W. (1999), Preparation and properties of totally synthetic immunogenes, Vaccine Volume 18, Issues 3-4, September 1999, pages 355-361; and Black, M., Trent, A., Tirrell, M. and Olive, C. (2010), Advances in the design and delivery of peptide subunit vaccines with a focus on Toll-like receptor agonists, Expert Rev Vaccines, 2010 February; 9(2): 157-173. In brief, the peptide should have as many of the following properties as possible: (a) it is highly hydrophilic, (b) it comprises at least one residue from the group of aspartate, proline, tyrosine and phenylalanine, (c) it has no homology or only a slight homology with other known peptides or polypeptides in order to achieve a high specificity, (d) it must be sufficiently soluble and (e) it does not comprise any glycosylation or phosphorylation sites, unless they are required for specific reasons. Alternatively, it is possible to use bioinformatic approaches, for example those described in Moreau, V., Fleury, C., Piquer, D., Nguyen, C., Novali, N., Villard, S., Laune, D., Granier, C. and Molina, F. (2008), PEPOP: Computational design of immunogenic peptides, BMC Bioinformatics 2008, 9:71.

The polypeptide according to the invention can be provided in any conformation. For example, the polypeptide can be substantially unfolded or partially or completely folded. In a preferred embodiment, the polypeptide is folded in the sense that the epitopes required for binding the antibody assume the fold which the native protein assumes in its natural environment. A person skilled in the art in the field is familiar with the methods which make it possible to determine whether a polypeptide is folded and, if said polypeptide is folded, which structure it assumes, for example limited proteolysis, NMR spectroscopy, CD spectroscopy or X-ray crystallography (see, for example, Banaszak L. J. (2008), Foundations of Structural Biology, Academics Press, or Teng Q. (2013), Structural Biology: Practical Applications, Springer).

For the purposes of purification or immobilization, the polypeptide according to the invention can have a tag, a sequence or domain which is capable of specifically binding to a ligand, for example a tag from the group comprising CBD, CBP, FLAG, c-Myc, Strep-tag, poly-Arg, His-tag, thioredoxin, MBP and GST.

The polypeptide according to the invention can be an immobilized polypeptide. In a preferred embodiment, the term “immobilized”, as used here, means a molecule bound to a solid support insoluble in aqueous solution, more preferably via a covalent bond, electrostatic interactions, encapsulation or trapping, for example by denaturation of a globular polypeptide in a gel, more preferably via hydrophobic interaction, most preferably via one or more than one covalent bond. Various suitable supports, for example paper, polystyrene, metal, silicone or glass surfaces, microfluidic channels, membrane, microbeads, such as magnetic microbeads, column chromatography media, biochips, polyacrylamide gels and the like, are described in the literature, for example in Kim et al. Protein immobilization techniques for microfluidic assays, Biomicrofluidics 7(4), 041501. This makes it possible to easily separate the immobilized molecule together with the insoluble support from an aqueous solution, for example by filtration, centrifugation or decanting. The immobilized molecule can be reversibly or irreversibly immobilized. For example, immobilization is reversible when the molecule interacts with the support via ionic interactions, which can be masked by addition of a high concentration of salt, or when the molecule is bound via a disengageable covalent bond, such as, for example, a disulfide bond which can be cleaved by addition of thiol-containing reagents. The polypeptide can be indirectly immobilized, for example by immobilization of an antibody or of a different molecule having an affinity for the polypeptide, followed by formation of a complex, with the effect that the polypeptide/antibody complex is immobilized. Various methods for immobilizing polypeptides are described in the literature, for example in Kim et al. Various reagents and kits for immobilization reactions are commercially available, for example from Pierce Biotechnology.

The invention provides a kit, preferably for diagnosing a disease. Such a kit can contain instructions for using the kit and a means for contacting the polypeptide according to the invention with a liquid body sample from a subject, preferably a human subject, for example a line blot, the polypeptide according to the invention being immobilized on the line blot. The performance of line blot-based tests and their design is described in the prior art (Raoult, D., and Dasch, G. A. (1989), The line blot: an immunoassay for monoclonal and other antibodies. Its application to the serotyping of gram-negative bacteria. J. Immunol. Methods, 125 (1-2), 57-65; WO2013041540).

Furthermore, the kit can comprise a positive control, for example an antibody known to bind to the polypeptide according to the invention, and a negative control, for example a protein having no detectable affinity for the polypeptide according to the invention, for example bovine serum albumin. Lastly, such a kit can comprise a standard solution of the antibody or polypeptide for calibration purposes.

The invention provides an antibody capable of specifically binding to the polypeptide according to the invention. In a preferred embodiment, the term “antibody”, as used here, means all immunoglobulin-based binding entities, more preferably at least one immunoglobulin heavy chain and one immunoglobulin light chain, encompassing, but not restricted to, monoclonal and polyclonal antibodies as well as variants of an antibody, more particularly fragments capable of binding to the antigen, preferably of binding highly specifically. In a particular embodiment, the term “specifically binding”, as used here, means that binding is stronger than a binding reaction characterized by a dissociation constant, especially a dissociation constant of 1×10⁻⁵ M, more preferably 1×10⁻⁷ M, more preferably 1×10⁻⁸ M, more preferably 1×10⁻⁹ M, more preferably 1×10⁻¹⁰ M, more preferably 1×10⁻¹¹ M, more preferably 1×10⁻¹² M, as determined by surface plasmon resonance using Biacore instruments at 25° C. in PBS buffer, pH 7. The antibody can be present isolated and in a mixture comprising further antibodies, polypeptides, metabolites, cells and the like.

The reagents, devices, methods and uses which are described in this application can be used for diagnosing a disease. In a preferred embodiment, said disease is an allergy. In a more preferred embodiment, the disease is an allergy to nuts, preferably to at least one nut from the group comprising hazel nuts, sesame, peanuts, walnuts and pecans. In a preferred embodiment, the term “diagnosis”, as used here, means any kind of procedure geared toward obtaining information which supports the assessment of whether a subject has suffered from a particular disease in the past, is suffering from a particular disease at the time of diagnosis or will suffer from a particular disease in the future, or that this is more likely than for an average comparative subject exhibiting similar clinical symptoms, or for finding out how the disease has progressed or is likely to progress in the future, or for evaluating the response of the patient to a particular type of treatment. In other words, the term “diagnosis” encompasses not only the diagnosis of a disease, but also the prognosis of a disease or the monitoring of the course of a disease.

In many cases, the mere detection of the antibody suffices, in other words the determination of whether detectable concentrations of the antibody are present in the sample. If the antibody is detected, this is a significant piece of information for the diagnosis by the care-providing physician and indicates an increased likelihood of the patient suffering from the disease. In a preferred embodiment, the relative concentration of the antibody in the serum can be compared with the concentration found in an average healthy subject. The method according to the invention can include determining whether the concentration of the antibody is at least 0.1, preferably 0.2, 0.5, 1, 2, 5, 10, 20, 25, 50, 100, 200, 500, 1000, 10 000 or 100 000 times higher than that concentration that would be found in an average healthy subject.

A person skilled in the art in the field acknowledges that the practicing treatment-providing physician does not make his/her diagnosis solely on the basis of an individual diagnostic parameter, but instead needs to take numerous aspects into account, for example the presence of other antibodies, markers, blood parameters, clinical investigation of the symptoms of the patient or the results of imaging or other noninvasive methods, in order to establish the overall diagnosis. The value of a diagnostic agent or of a diagnostic method can also be to rule out one or more diseases and to thereby permit the indirect diagnosis of another disease. In a preferred embodiment, the meaning of all symptoms or diseases to which reference is made in this application is to be considered in accordance with the understanding of a person skilled in the art on the filing date, as described by textbooks and scientific publications on said date.

Thus, the term “diagnosis” preferably does not mean that the diagnostic methods or reagents according to the invention will yield a definitive and sufficient result. Definitively making the diagnosis on the basis of an individual test, or even parameter, can mean a contribution to a “differential diagnosis”, i.e., to a systematic diagnostic procedure which considers the likelihood of a range of diseases on the basis of a range of diagnostic parameters. The term “diagnosis” can also mean that a method or reagent is used in order to distinguish between two or more diseases with similar symptoms.

The term “diagnosis” can also mean determining which reagent or treatment method from two or more available options is the most promising for the patient.

The polypeptide according to the invention can be used in a combination with further allergy-specific antigens. In this connection, it is possible to use a mixture or a combination comprising multiple spatially separated and purified antigens, preferably in immobilized form, even more preferably on a medical device such as a line blot. By combining various isolated allergens, it is possible to establish an individual sensitization pattern for every patient and to thus obtain information on the severity of the allergy present or else on the future course of the disease.

A polypeptide according to the invention comprising an antigenic fragment having a sequence of at least seven amino acids from a hydrophilic domain from a peanut oleosin, peanut caleosin or peanut steroleosin, preferably from a peanut oleosin, can be combined with one or more than one allergen or variant thereof from the group comprising Ara h 1 (P43238), Ara h 2 (Q6PSU2), Ara h 6 (Q647G9), Ara h 9 (B6CG41), more preferably from the group comprising Ara h 1, Ara h 2, Ara h 6, even more preferably Ara h 2 and Ara h 6, most preferably with Ara h 2.

A polypeptide according to the invention comprising an antigenic fragment having a sequence of at least seven amino acids from a hydrophilic domain from a hazelnut oleosin, hazelnut caleosin or hazelnut steroleosin, preferably from a hazelnut oleosin, can be combined with one or more than one allergen or variant thereof from the group comprising Cor a 1 (Q9SWR4), Cor a 2 (Q9AXH5), Cor a 8 (Q9ATH2), Cor a 9 (Q8W1C2) and Cor a 14 (DOPWG2), more preferably from the group comprising Cor a 1, Cor a 8, Cor a 9 and Cor a 14, more preferably Cor a 1, Cor a 9 and Cor a 14, even more preferably Cor a 9 and Cor a 14, most preferably with Cor a 14.

A polypeptide according to the invention comprising an antigenic fragment having a sequence of at least seven amino acids from a hydrophilic domain from a sesame oleosin, sesame caleosin or sesame steroleosin, preferably from a sesame oleosin, can be combined with one or more than one allergen or variant thereof from the group comprising Ses i 1 (Q9AUD1), Ses i 2 (Q9XHP1), Ses i 3 (Q9AUD0), Ses i 6 (Q9XHP0) and Ses i 7 (Q9AUD2), more preferably from the group comprising Ses i 1, Ses i 2, Ses i 3 and Ses i 6, even more preferably Ses i 1, Ses i 2, and Ses i 3, even more preferably Ses i 1, Ses i 2, most preferably with Ses i 2.

In a preferred embodiment, the detection of the complex is carried out using a method from the group comprising immunodiffusion techniques, immunoelectrophoretic techniques, light-scattering immunoassays, agglutination techniques, labeling techniques, such as from the group comprising radioimmunoassay, enzyme immunoassay, chemiluminescence immunoassay and immunofluorescence techniques. A person skilled in the art in the field is familiar with said methods, which are also described in the prior art, for example in Zane, H. D. (2001), Immunology—Theoretical & Practical Concepts in Laboratory Medicine, W. B. Saunders Company, especially in chapter 14.

The step “contacting a liquid sample comprising antibodies with the polypeptide according to the invention” is carried out under suitable conditions. In a preferred embodiment, the term “conditions compatible with the formation of the complex” means conditions allowing the specific antigen/antibody interactions to establish the complex comprising the polypeptide and the antibody. In a preferred embodiment, such conditions can comprise the incubation of the polypeptide at 25° C. for 30 minutes in a sample diluted 1:100 in PBS buffer. Any data, including presence or absence of the complex comprising the antibody and the polypeptide according to the invention, can be correlated with reference data. For example, the detection of the complex can indicate that the patient from whom the sample originates has suffered from a disease or will suffer from a disease in the future. If the patient has been diagnosed before and the diagnostic method is carried out again, the amount of complex detected in the two runs can be correlated in order to evaluate the progression of the disease and/or the success of treatment. If the complex amount increases, for example, this suggests that the disease is progressing and is likely to manifest itself in the future and/or that the treatment is not successful.

The sample which is being used for the diagnostic method according to the invention must contain antibodies, which are also referred to as immunoglobulins. Typically, the sample comprises a representative amount of all the antibodies of the subject to be examined. After it has been provided, the sample can, however, also be processed, and this can comprise a fractionation, centrifugation, enrichment or isolation of all the antibodies or of a particular antibody class, and this can influence the relative distribution of immunoglobulin of different classes such that it deviates from the original distribution. In a preferred embodiment, class IgE immunoglobulins are enriched.

The therapeutic polypeptide can be used therapeutically or for producing a medicament, for example a reagent for the purposes of desensitization or as a vaccine. In this connection, said polypeptide is in a pharmaceutically acceptable form, for example in combination with a carrier.

The antibody according to the invention or antibody detected according to the invention is an IgE class antibody or IgG class antibody, for example IgG4, particularly preferably of the IgE class. A person skilled in the art in the field is familiar with techniques for purifying antibodies, for example those described in Hermanson et al.

This application and the accompanying sequence listing refer to a range of sequences, specifically:

| SEQ ID NO | Description |
|---|---|
| SEQ ID NO: 1 | N-terminal hydrophilic domain of the oleosin |
| | from hazelnut |
| SEQ ID NO: 2 | C-terminal hydrophilic domain of the oleosin |
| | from hazelnut |
| SEQ ID NO: 3 | N-terminal hydrophilic domain of the oleosin |
| | from walnut |
| SEQ ID NO: 4 | C-terminal hydrophilic domain of the oleosin |
| | from walnut |
| SEQ ID NO: 5 | Consensus sequence I of oleosins |
| SEQ ID NO: 6 | Consensus sequence II of oleosins |
| SEQ ID NO: 7 | C-terminal sequence motif I of oleosins |
| SEQ ID NO: 8 | C-terminal sequence motif II of oleosins |
| SEQ ID NO: 9 | Consensus sequence I for hydrophobic domain of |
| | oleosins |
| SEQ ID NO: 10 | Consensus sequence II for hydrophobic domain of |
| | oleosins |
| SEQ ID NO: 11 | Consensus sequence I of caleosins |
| SEQ ID NO: 12 | Consensus sequence II of caleosins |
| SEQ ID NO: 13 | Consensus sequence I for hydrophobic domain of |
| | caleosins |
| SEQ ID NO: 14 | Consensus sequence II for hydrophobic domain of |
| | caleosins |
| SEQ ID NO: 15 | Consensus sequence I of steroleosins |
| SEQ ID NO: 16 | Consensus sequence II of steroleosins |
| SEQ ID NO: 17 | Consensus sequence I for hydrophobic domain of |
| | steroleosins |
| SEQ ID NO: 18 | C-terminal sequence motif I of steroleosins |
| SEQ ID NO: 19 | C-terminal sequence motif II of steroleosins |
| SEQ ID NO: 20 | C-terminal sequence motif III of steroleosins |
| SEQ ID NO: 21 | C-terminal sequence motif IV of steroleosins |
| SEQ ID NO: 22 | Exemplary linker sequence |
| SEQ ID NO: 23 | Exemplary construct derived from Cor a |
| SEQ ID NO: 24 | Sequence of Ara h 15 with N-terminal His-Tag |
| SEQ ID NO: 25 | Sequence of Ara h 15 with N-terminal His-Tag without |
| | hydrophobic domain |

The invention will be elucidated below by means of exemplary embodiments with reference to the figures. The described embodiments are to be understood in all respects as merely exemplary and not as limiting, and various combinations of the cited features are covered by the scope of the invention.

### Example 1

### Cloning, Recombinant Expression and Purification of Cor a 12

The coding region of the modified Cor a 12 (SEQ ID N0:23) was generated by gene synthesis (Eurofins Genomics GmbH), amplified by polymerase chain reaction and, using the pBAD/TOPO® ThioFusion™ Expression Kit (Invitrogen), subcloned into the recombinant vector. The fusion protein was expressed in E. coli according to manufacturer's instructions and purified by affinity chromatography by means of a nickel matrix (IMAC).

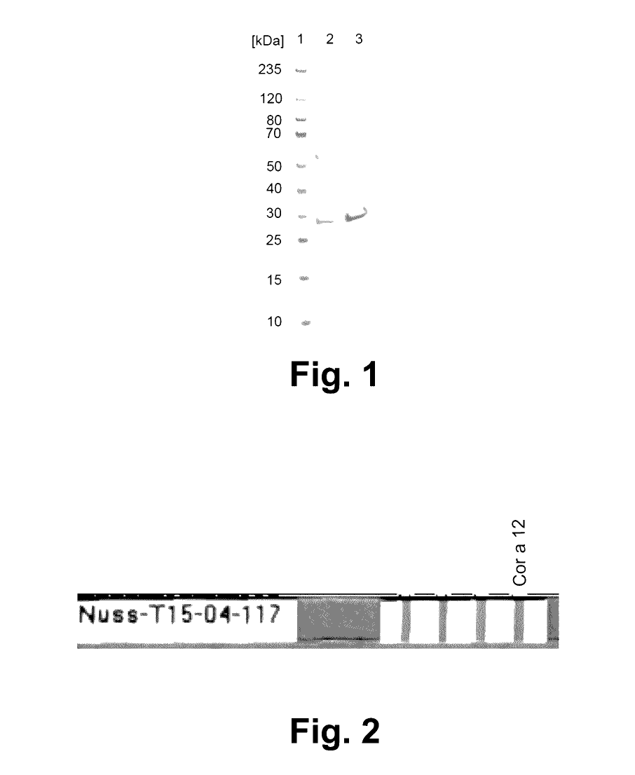

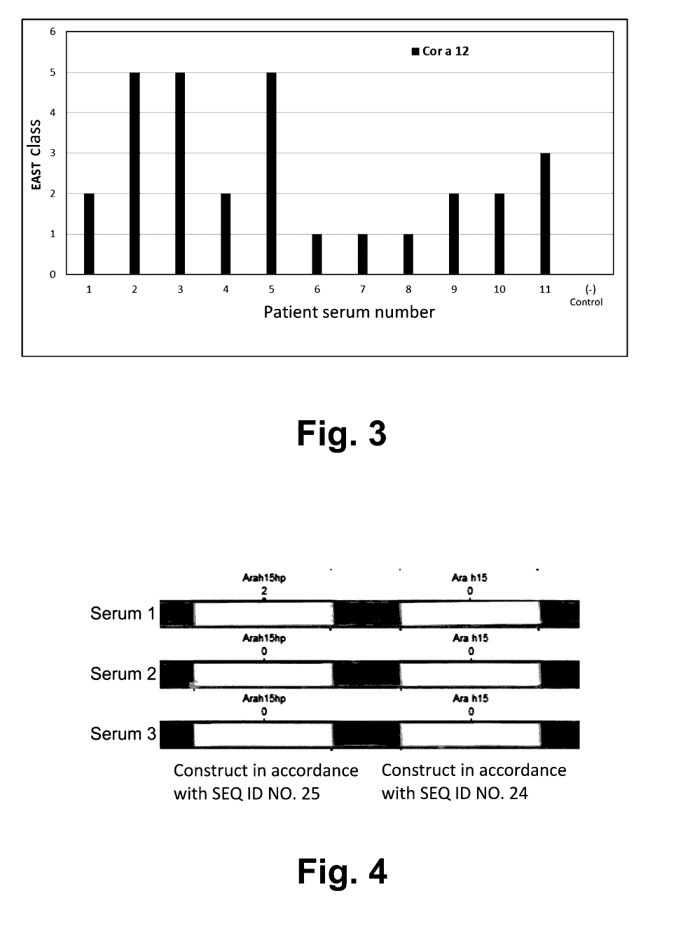

The recombinant construct was analyzed by SDS-PAGE; FIG. 1 shows the Coomassie-stained SDS gel. 3 μg of protein were loaded per lane.

### Example 2

### Preparation of a Line Blot (EUROLINE)

For the purpose of preparing the Euroline, the proteins according to the invention were directly coated onto a nitrocellulose membrane (Schleicher & Schüll) by means of a precision dispenser (Zeta Corporation). In this process, homogeneous lines are drawn. The membranes were subsequently blocked according to manufacturer's instructions and washed and cut into 3 mm wide strips.

### Example 3

### Performance of the Test According to the Invention

Each strip was contacted separately with 1 ml of the patient sample diluted 1:10 (v/v) in wash buffer (20 mM Tris, pH 7.5) and incubated at room temperature for 16 h; the serum was removed and the strip was then washed 3 times with 1 ml of wash buffer for 5 min each time. After the wash buffer had been poured off, each strip was incubated at room temperature with 1 ml of anti-human IgE antibody, AP-conjugated (Abcam, diluted 1:1000 (v/v) in wash buffer). After one hour, the conjugate was discarded, the strips were again washed three times with 1 ml of wash buffer and incubated with BCIP/NBT substrate for 10 minutes, and the reaction was lastly stopped using distilled water.

Evaluation was carried out by means of a scanner and the EUROLINEScan® program (EUROIMMUN). A semiquantitative assessment can be carried out according to WHO standard (75/502) by means of an IgE calibration curve. Concentration is given in the international unit kU/L (kilo IgE units per liter). An example of a reference system available for the interpretation of the result is the RAST system (radioallergosorbent test), which is widespread in allergy diagnostics and was developed by Pharmacia (now Thermo Scientific) (Buhl et al., “Allergologie—Handbuch” [Allergology—a manual], edited by K. Mohr. Stuttgart: Schattauer 2011). The EUROLINE-Scan program automatically converts the Euroline band intensities into EAST (enzyme allergosorbent test) classes, which, with regard to the concentration gradations, correspond to the RAST system. The division of the EAST (enzyme allergosorbent test) classes is shown in Tab. 1:

TABLE 1
EAST classes
EAST classes

| EAST class | Concentration [kU/L] |
|---|---|
| 0 | <0 35 |
| 1 | 0.35-0.7 |
| 2 | 0.7-3.5 |
| 3 | 3.5-17.5 |
| 4 | 17.5-50 |
| 5 | 50-100 |
| 6 | >100 |

The patient sera used for the Euroline with Cor a 12 were precharacterized sera from hazelnut allergy sufferers. For the negative control, no serum was incubated.

### Example 4

### Comparative Experiments Based on Conventional Ara h 15 Antigen and Ara h 15 Having a Truncated Hydrophobic Domain According to the Present Invention

The coding regions of the non-modified (SEQ ID NO:24) and the modified Ara h 15 (SEQ ID NO:25) were generated by means of gene synthesis (Eurofins Genomics GmbH), amplified by polymerase chain reaction and subcloned into the recombinant pBAD/TOPO® vector using the ThioFusion™ Expression Kit (Invitrogen). The constructs expressed in E. coli were purified by affinity chromatography by means of a nickel matrix (NICKEL RAPID RUN, ABT Agarose Bead Technologies).

The constructs prepared in this manner were coated onto a nitrocellulose membrane as described in Example 2 and incubated as described in Example 3. A serum from a patient with clinical symptoms upon consumption of nuts (Serum 1) as well as two sera from allergy sufferers without specific class E immunoglobulins against nuts (Serum 2 and 3) were used. The evaluation was conducted as described in Example 3 by means of the quoted EUROLineScan program.

FIG. 4 shows that IgE antibodies to Ara h 15 could only be detected in patient 1 if an Ara h 15 construct having a truncated hydrophobic domain was used to capture IgE antibodies. By contrast, an assay using full-length Ara h 15 yielded a false-negative result. No IgEs could be detected in patients suffering from allergies other than nut allergies. This shows that the assay according to the invention is specific and more sensitive that state of the art assays.

The various embodiments described above can be combined to provide further embodiments. All of the U.S. patents, U.S. patent application publications, U.S. patent applications, foreign patents, and foreign patent applications listed in the Application Data Sheet are incorporated herein by reference in their entirety.

In addition, all the other U.S. patents, U.S. patent application publications, U.S. patent applications, foreign patents, foreign patent applications, and non-patent references cited in the present disclosure, including sequences represented by database codes specifically cited in the present disclosure, are also incorporated herein by reference if they do not conflict or contradict with other descriptions of the present disclosure.

1. A polypeptide comprising an antigenic fragment having a sequence of at least seven amino acids from a hydrophilic domain of an oil body-associated protein,
wherein the polypeptide comprises a truncated hydrophobic domain of the oil body-associated protein or does not comprise such a domain, and
wherein the oil body-associated protein is selected from the group comprising oleosin, caleosin and steroleosin.

2. The polypeptide as claimed in claim 1, comprising
a) an antigenic fragment of at least seven amino acids from an N-terminal hydrophilic domain of an oil body-associated protein and
b) an antigenic fragment of at least seven amino acids from a C-terminal hydrophilic domain of an oil body-associated protein,
wherein a) and b) are fused to one another or are joined to one another by means of a linker, and
wherein the oil body-associated protein is selected from the group comprising oleosin and caleosin.

3. The polypeptide as claimed in claim 1,
wherein the oil body-associated protein is an oleosin.

4. The polypeptide as claimed in claim 2, wherein a) is a sequence from SEQ ID NO:1 and variants thereof and b) is a sequence from SEQ ID NO:2 and variants thereof.

5. The polypeptide as claimed in claim 2, wherein a) is a sequence from SEQ ID NO:3 and variants thereof and b) is a sequence from SEQ ID NO:4 and variants thereof.

6. The polypeptide as claimed in claim 1, wherein the polypeptide is immobilized, preferably on a solid support, even more preferably on a microbead.

7. A pharmaceutical composition or a vaccine comprising the polypeptide as claimed in claim 1.

8. A complex comprising the polypeptide as claimed in claim 1 and an antibody, preferably of the IgE class, bound thereto.

9. A medical or diagnostic device, comprising the polypeptide as claimed in claim 1.

10. The medical or diagnostic device as claimed in claim 9, wherein said device is a test strip, a biochip or an electrophoresis gel.

11. A nucleic acid encoding the polypeptide as claimed in claim 1 or a cell comprising said nucleic acid.

12. A test kit comprising the polypeptide as claimed in claim 1, optionally additionally a means for detecting the complex that comprises the polypeptide as claimed in claim 1 and an antibody, preferably of the IgE class, bound thereto.

13. A method comprising the step of detecting an antibody which binds to the polypeptide as claimed in claim 1 in a sample from a mammal, preferably a human, containing antibodies, preferably of the IgE class.

14. The method as claimed in claim 13, wherein the mammal exhibits at least one symptom of an allergy and/or it is suspected that the mammal suffers from an allergy, preferably to nuts.

15. The method as claimed in claim 13, comprising the steps of
a) providing a sample from a mammal,
b) contacting the sample with the polypeptide or a medical or diagnostic device comprising the polypeptide under conditions compatible with the formation of a complex between the polypeptide and an antibody,
and
c) detecting a complex formed in step b).
